(19) United States
(12) Patent Application Publication
(10) Pub. No.: US 2013/0267838 A1
(43) Pub. Date: Oct. 10, 2013

(54) Augmented Reality System for Use in Medical Procedures

(71) Applicant: Board of Regents, the University of Texas System, Galveston, TX (US)

(72) Inventors: Bennjamin D. Fronk, Dickinson, TX (US); Daneshvari R. Solanki, League City, TX (US); Varun Koyyalagunta, Austin, TX (US)

(73) Assignee: Board of Regents, the University of Texas System, Galveston, TX (US)

(21) Appl. No.: 13/857,851

(22) Filed: Apr. 5, 2013

Related U.S. Application Data
(60) Provisional application No. 61/621,740, filed on Apr. 9, 2012.

(51) Int. Cl. A61B 5/06; A61B 5/00

(52) U.S. Cl. CPC A61B 5/066; A61B 5/064; A61B 5/0077; A61B 5/7425; A61B 5/72; A61B 5/489
USPC 600/424

(57) ABSTRACT

An augmented realty system is disclosed that allows a clinician to create and view a 3D model of structure of interest using an imaging device prior to introduction of a tool designed to interact with that structure. The 3D model of the structure can be viewed by the clinician through a head mounted display (HMD) in its proper position relative to the patient. With the 3D model of the structure in view, the imaging device can be dispensed with, and the clinician can introduce the tool into the procedure. The position of the tool is likewise tracked, and a virtual image of a 3D model of the tool is also viewable through the HMD. With virtual images of both the tool and the structure in view, the clinician can visually verify, or a computer coupled to the HMD can automatically determine, when the tool is proximate to the structure.

### CROSS-REFERENCE TO RELATED APPLICATIONS

This is a non-provisional of U.S. Provisional Patent Application Ser. No. 61/621,740, filed Apr. 9, 2012, to which priority is claimed, and which is incorporated herein by reference.

### FIELD OF THE INVENTION

This disclosure relates to an augmented reality system useful in a medical procedure involving interaction between a structure of interest in a patient and a tool.

### BACKGROUND

Imaging is important in medical science. Various forms of imaging, such as ultrasound, X-ray, CT scan, or MRI scans, and others, are widely used in medical diagnosis and treatment.

FIG. 1 illustrates one use of imaging in a medical procedure. In this example, it is desired to insert a tool 27 having a needle 26 into a vessel 24 below a patient's skin 22. This may be necessary for the placement of a central line in the patient for the administration of intravenous (IV) fluids and medications, in which case the needle 26 would eventually be removed from the tool 27 after placement and its catheter connected to an IV line.

Because the vessel 24 may be deep below the skin 22 and therefore not visible to a clinician (e.g., doctor), it can be helpful to image the vessel 24, and in FIG. 1 such imaging is accomplished through the use of an ultrasound device 12. As is well known, the ultrasound device 12 includes a transducer or probe 18 coupled to the device by a cable 16. The transducer 18, under control of the ultrasound 12, emits sound waves in a plane 20, and reports reflections back to the ultrasound, where the image of the vessel 24 can be displayed on a screen 14. If the needle 26 is introduced into the patient along the plane 20 of the transducer 18, then the image of the needle, and in particular its tip 28, will also be visible in the display 14 in real time. In this way, the clinician can view the screen 14 to verify the position of the needle tip 28 relative to the vessel 24, and particularly in this example can verify when the needle tip 28 has breached the wall of the vessel 24.

While ultrasound imaging is helpful in this procedure, it is also not ideal. The clinician must generally look at the ultrasound screen 14 to verify correct positioning of the needle tip 28, and thus is not looking solely at the patient, which is generally not preferred when performing a medical procedure such as that illustrated. Additionally, the ultrasound transducer 18 must be held in position while the needle 26 is introduced, either by the clinician (with a hand not holding the tool 27) or by another clinician present in the procedure room. The technique illustrated in FIG. 1 is thus either a two-man procedure, with one clinician holding the tool 27 and the other the transducer 18, or a cumbersome one-man procedure in which the clinician must hold both. Care must also be taken to align the plane 20 of the transducer with the axis of the needle 26 so that it can be seen along its length. If the plane 20 crosses the needle axis at an angle, the needle would be imaged only as a point, which may not be resolvable on the screen 14 and which may otherwise be unhelpful in determining the position of the needle tip 28 relative to the vessel 24.

This is but one example showing that imaging during a medical procedure, while helpful, can also be distracting to the task at hand. Other similar examples exist. For example, instead of a vessel 24, a structure of interest may comprise a tumor, and the tool 27 may comprise an ablating tool or other tool for removing the tumor. Again, imaging can assist the clinician with correct placement of the ablating tool relative to the tumor, but the clinician is distracted by simultaneously dealing with the tool and the imaging device.

The inventors believe that better solutions to problems of this nature are warranted and have come up with solutions.

### BRIEF DESCRIPTION OF THE DRAWINGS

FIG. 1 illustrates use of an imaging device (ultrasound) to help position a tool (needle) in a structure of interest (vessel) in accordance with the prior art.

FIG. 2 illustrates one example of an improved system to help position a tool proximate to a structure of interest, using augmented reality and optical markers to assess relative positions of components in the system.

FIGS. 3A and 3B illustrate an initial step in the process in which a patient marker is optically tracked using a head mounted display (HMD).

FIGS. 4A and 4B illustrate a next step in which an ultrasound transducer marker is additionally optically tracked using the HMD.

FIGS. 5A-5C illustrate a next step in which the transducer is used to form a virtual 3D image of the structure of interest.

FIGS. 6A and 6B illustrate a next step in which the transducer is removed, and the virtual 3D image of the structure of interest is viewed through the HMD.

FIGS. 7A and 7B illustrate a next step in which in which a tool is introduced, in which a tool marker is additionally optically tracked using the HMD, and in which a virtual 3D image of the tool is displayed through the HMD.

FIGS. 8A and 8B illustrate a next step during which the tool is inserted in the patient, and a collision between the tool and structure of interest can be visually verified, and automatically verified with the computer.

FIG. 9 illustrates another example of an improved system to help position a tool proximate to a structure of interest, using augmented reality and optical markers to assess relative positions of components in the system, in which the camera is separated from the head mounted display.

FIG. 10 illustrates an initial step in the process in which a patient marker is optically tracked in the system of FIG. 9.

### DETAILED DESCRIPTION

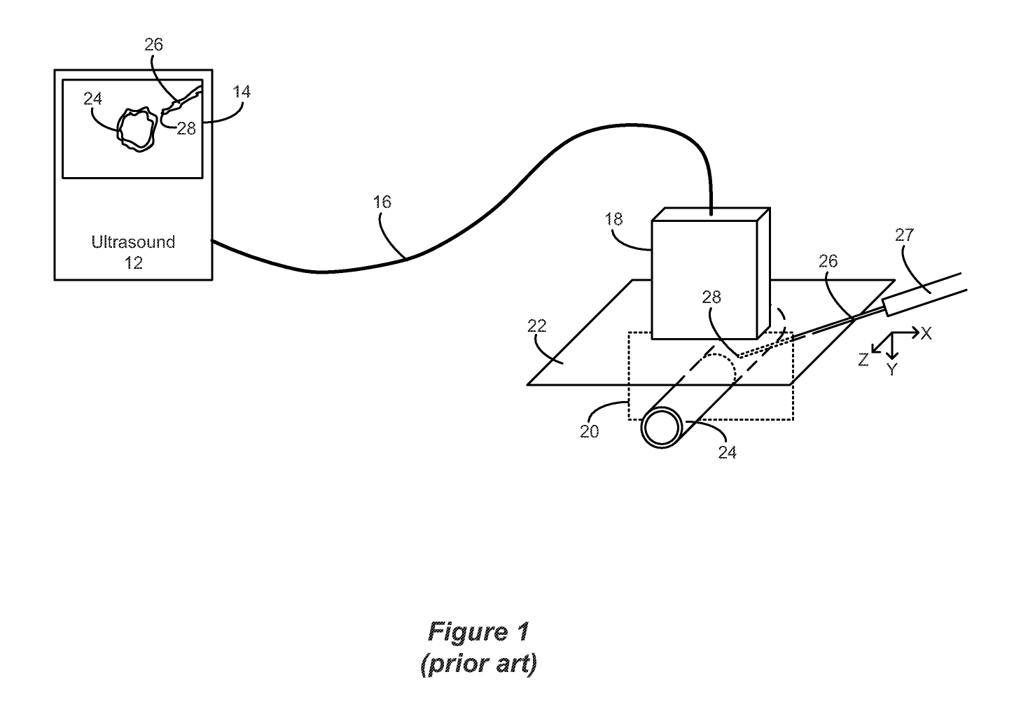

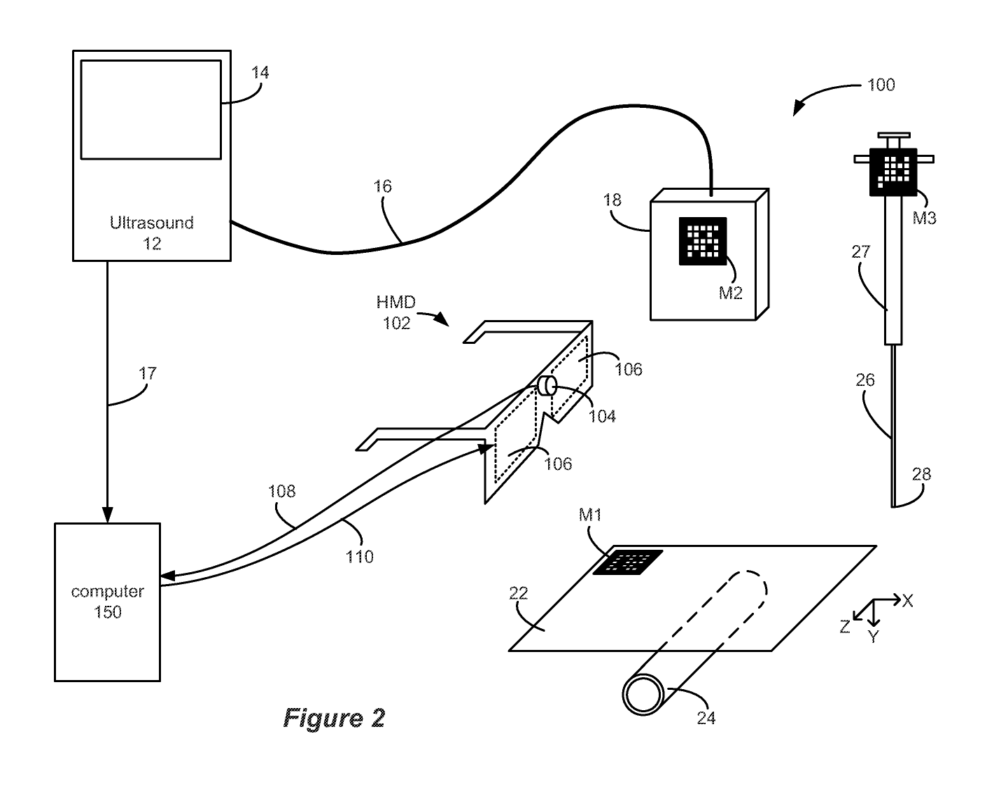

FIG. 2 shows an example of an improved augmented reality system 100 for imaging a structure of interest while performing a medical procedure involving a tool. The same example provided in FIG. 1 is again illustrated: placement of a needle 26 within a vessel 24 as assisted by ultrasound imaging. Thus, several similar elements are once again shown, including the ultrasound device 12, its transducer 18, the tool 27 including the needle 26, and the vessel 24 under the skin 22 of the patient. New to the system 100 are a computer 150, a head mounted display (HMD) 102, and several markers (M1, M2, and M3). Marker M1 is affixed to the patient's skin 22, marker M2 is affixed to the ultrasound transducer 18, and marker M3 is affixed to the tool 27.

By way of an overview, the system 100 allows the clinician to create a 3-dminesional (3D) model of the vessel 24 using the ultrasound 12. This 3D model, once formed, can be viewed by the clinician through the HMD 102 in its proper position relative to the patient. That is, through the HMD 102, the clinician can see both a virtual image of the 3D model of the vessel 24 superimposed on the clinician's view, such that the 3D model of the vessel will move and retain its correct position relative to the patient when either the clinician or patient moves. With the 3D model of the vessel in view, the ultrasound 12 can now be dispensed with, and the clinician can introduce the tool 27 into the procedure. The position of the tool 27 is likewise tracked, and a virtual image of a 3D model of the tool 27 is also superimposed in the HMD 102 onto the clinician's view along with the 3D model of the vessel 24.

With both 3D models for the vessel 24 and tool 27 visible through the HMD 102, the clinician can now introduce the tool 27 into the patient. As the clinician virtually sees both the needle tip 28 of the tool 27 and the 3D model of the vessel 24 through the HMD 102, the clinician can visually verify when the tip 28 is proximate to, or has breached, the vessel 24. Additionally, because the positions of the 3D models are tracked by the computer 150, the computer 150 can also inform the clinician when the tool 27 and vessel 24 collide, i.e., when the tip 28 is proximate to, or has breached, the vessel 24. Beneficially, the clinician is not bothered by the distraction of imaging aspects when introducing the tool 27 into the patient, as the ultrasound 12 has already been used to image the vessel 24, and has been dispensed with, prior to introduction of the tool 27. There is thus no need to view the display 14 or manipulate the transducer 18 of the ultrasound during introduction of the tool 27.

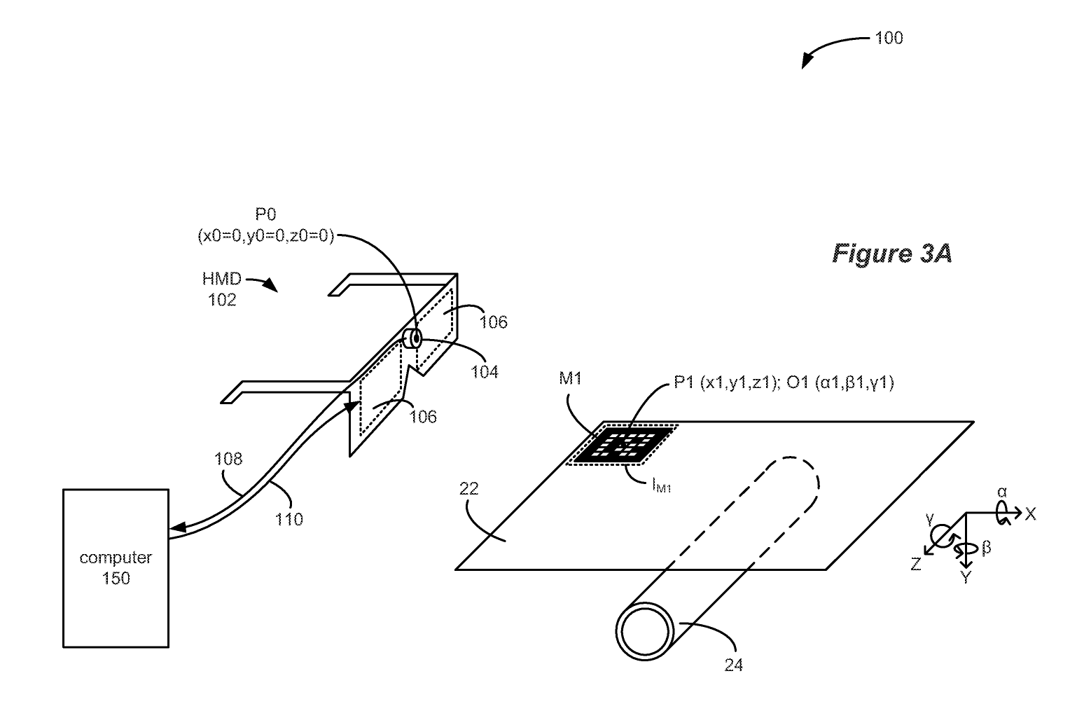

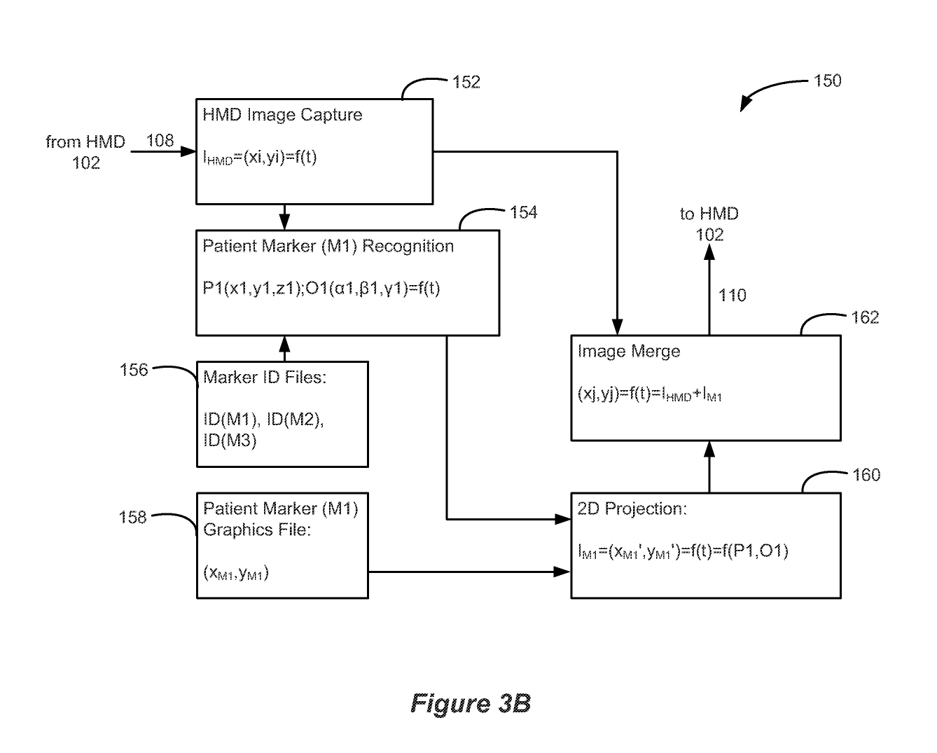

Different phases of the above-described procedure are set forth in subsequent figures, starting with FIGS. 3A and 3B. FIG. 3A shows the components of the system 100 used in an initial step. As shown, the system 100 at this point comprises the patient as represented by her skin 22 and the vessel 24 of interest, and a clinician (not shown) wearing the HMD 102. The HMD 102 comprises a camera 104 which sends live images to the computer 150 via cables 108. Further details of the processes occurring in the computer are shown in FIG. 3B, and these live images, h_{IIMD}, are seen in box 152 as a number of pixels (xi,yi) as a function of time (f(t)). Typically, optical capture of this sort comprises capturing a number of image frames at a particular frame rate, as one skilled in the art will understand. These live images I_{HMD} can be processed as necessary in the computer 150 and output back to the HMD 102 via cables 110 to displays 106 in the HMD 102 (FIG. 3A). Typically, there are two opaque displays in the HMD 102, one for each eye, although there may also be a single display viewable by both eyes in HMDs designs that are more akin to helmets rather than glasses. Regardless, the clinician sees the lives images as output by the display(s) 106. Such means of using a HMD 102 to view the real world is typical, and the HMD 102 can be of several known types. The HMD 102 may also be an optical see through type, again as is well known. In this modification, the displays 106 are at least semi-transparent, and as such live images don't need to be captured by the camera 104 and sent to the displays 106.

As discussed above, a marker M1 has been affixed to the patient's skin 22 in the vicinity of the vessel 24. The marker M1 in this example is encoded using a unique 2D array of black and white squares corresponding to a particular ID code (ID(M1)) stored in a marker ID file (box 156, FIG. 3B) in the computer 150. The marker M1 is recognized from the live images I_{HMD} in the computer 150, and its position P1(x1,y1,z1) and orientation O1(α1,β1,γ1) (i.e., how the marker M1 is turned with respect to the x, y, and z axes) relative to the camera 104 is determined by an optical analysis of the size and geometry of the squares in the marker M1 (box 154, FIG. 3B). Thus, the HMD 102, or more specifically the camera 104, acts as the origin of the system 100, whose position is understood by the computer 150 as P0 (x0=0,y0=0,z0=0). This means of optically determining the position and orientation of a structure using a marker is well known, and can be accomplished for example using ARToolKit or ArUco, which are computer tracking software for creating augmented reality applications that overlay virtual imagery on the real world. See “ARToolKit,” and “ArUco: a minimal library for Augmented Reality applications based on OpenCv,” which were submitted with the above-incorporated '740 Application.

Once marker M1 is recognized in the computer 150, it is beneficial to provide a visual indication of that fact to the clinician through the HMD 102. Thus, a 2-dimensional (2D) virtual image of the marker Ml, I_{M1}, is created and output to the displays 106. This occurs in the computer 150 by reading a graphical file of the marker (comprised of many pixels (x_{M1}, y_{M1}), and creating a 2D projection of that file (x_{M1}′,y_{M1}′). As shown in box 160, this image I_{M1} of marker M1 is a function of both the position P1 and orientation O1 of the marker M1 relative to the camera 104. Accordingly, as the clinician wearing the HMD 102 moves relative to the patient, the virtual image marker M1 image will change size and orientation accordingly. Software useful in creating 2D projections useable in box 160 includes the Panda3D game engine, as described in “Panda3D,” which was submitted with the above-incorporated '740 Application. Shading and directional lighting can be added to the 2D projections to give them a more natural look, as is well known.

In box 162, it is seen that the virtual images of the marker M1, I_{M1}, and the live images, I_{IIMD}, are merged, and output to the displays 160 via cables 110. Thus, and referring again to FIG. 3A, the clinician through the HMD 102 will see both live images and the time-varying virtual image of the marker, I_{M1}, which, like other images in the Figures that follow, is shown in dotted lines to reflect its virtual nature. Again, displaying the marker virtually is useful to inform the clinician that the marker has been recognized by the computer 150 and is being tracked. However, this is not necessary; other means informing the clinician of the recognition and tracking of the marker are possible using any peripherals typically used with computer 150 (not shown), such as sounds through speakers, indication on a computer system display, etc. Additionally, some other graphical indication of tracking can be superimposed on the displays 106 of the HMD 102.

Rendering a proper 2D projection that will merge with what the clinician is seeing through the HMD 102 typically involves knowledge of the view angle of the camera 104. Although not shown, that angle is typically input into the 2D projection module 160 so that the rendered 2D images will match up with the live images in the displays 106.

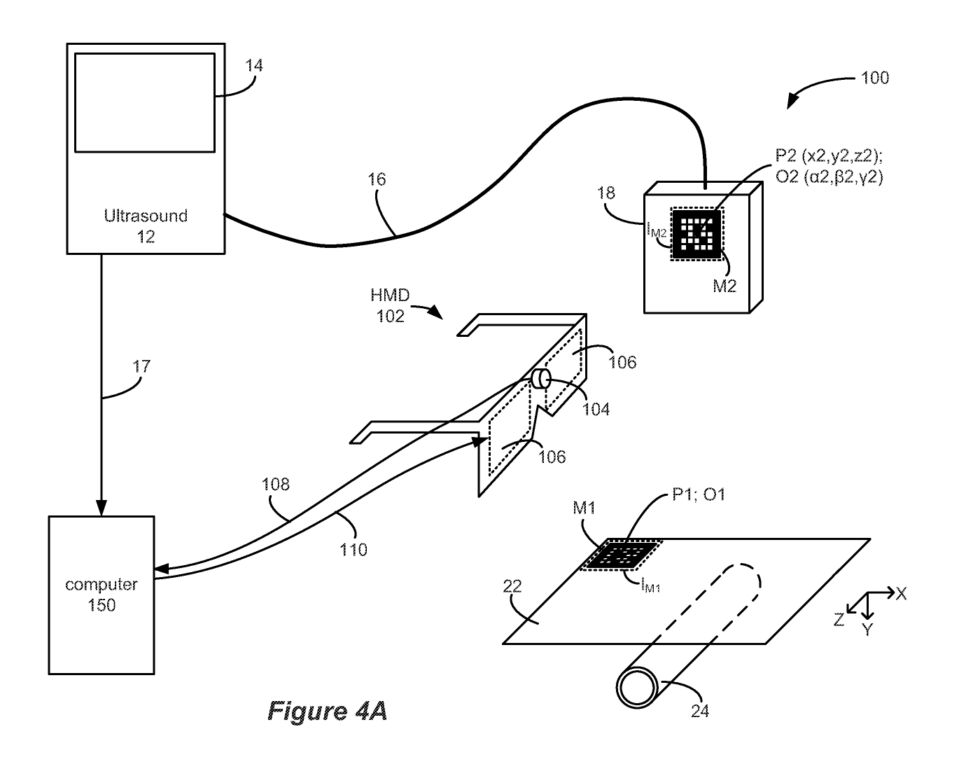

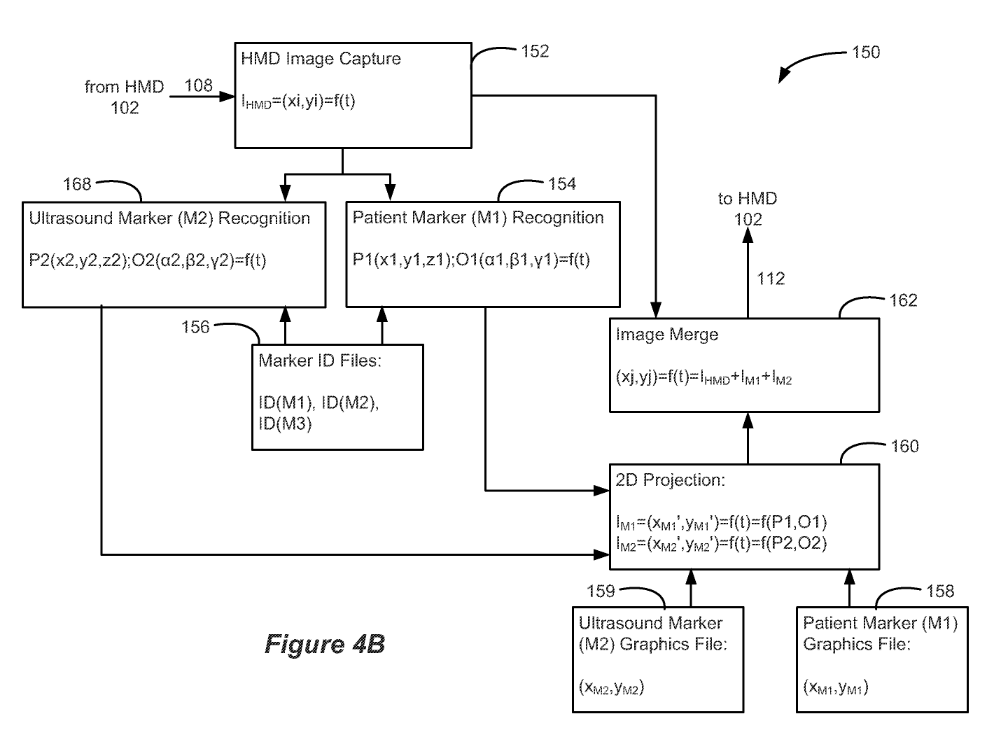

FIGS. 4A and 4B illustrate a next step, in which the ultrasound transducer 18 is introduced. A similar optically-detectable marker M2 is attached to the transducer 18 with its own unique ID code (ID(M2)) encoded in its pattern of squares. As with the patient marker M1, the position P2(x2,y2,z2) and orientation O2(α2,β2,γ2) of the transducer marker M2 relative to the camera 104 are recognized by the computer 150 (box 168, FIG. 4B). And again as with the patient marker, a 2D virtual image of the marker M2, I_{M2}, is created and output to the displays 106 by reading a graphical file of the marker (x_{M2}, y_{M2}), and creating a 2D projection (x_{M2}′,y_{M2}′) (boxes 159, 160). This virtual image I_{M2} of marker M2 is a function of both the position P2 and orientation O2 of the transducer marker M2 relative to the camera 104, and like image I_{M1} will change size and orientation as the HMD 102 moves. Merging of the transducer marker image I_{M2} with both the patient marker image I_{M1} and the live images I_{HMD} (box 162) lets the clinician know that the transducer is tracked, and that imaging of the vessel 24 can commence.

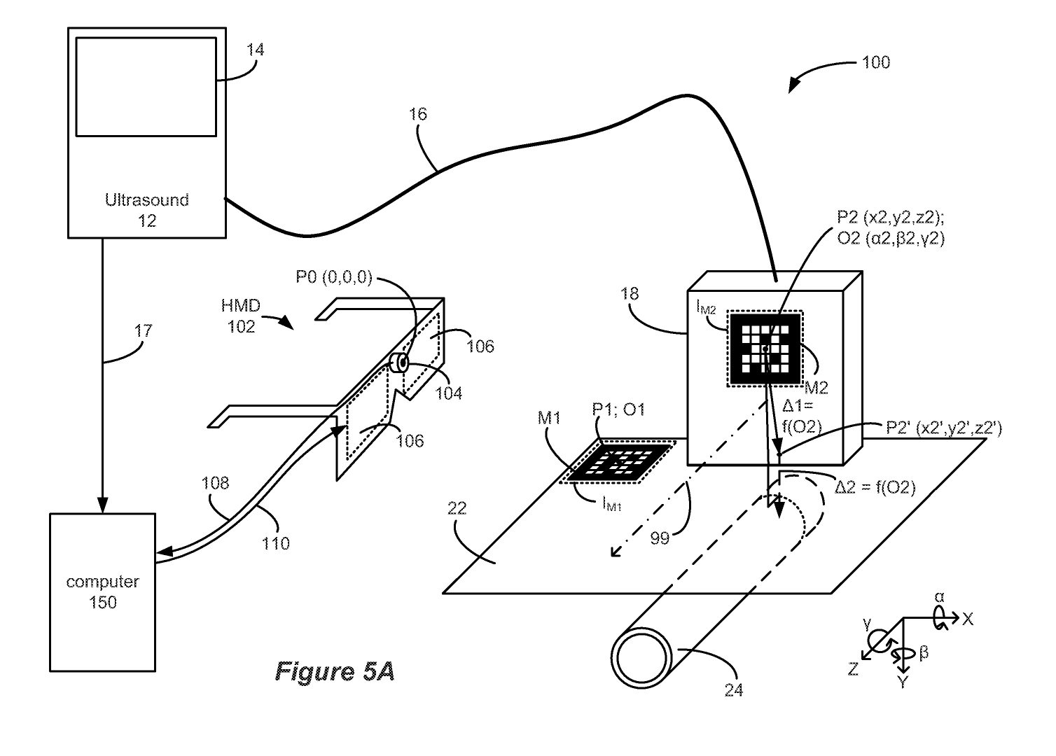

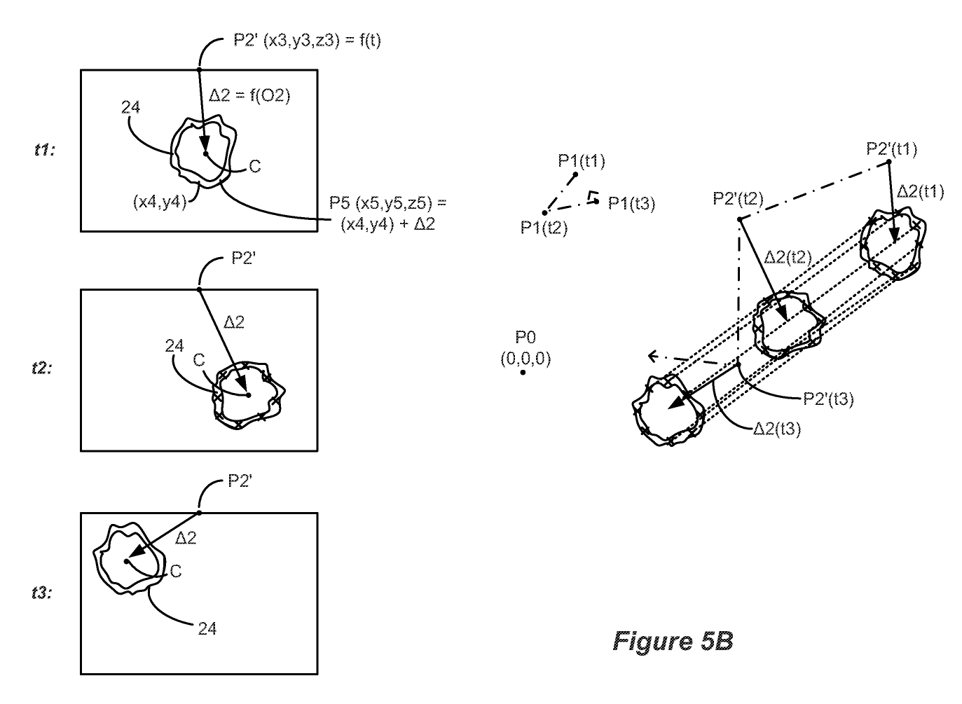

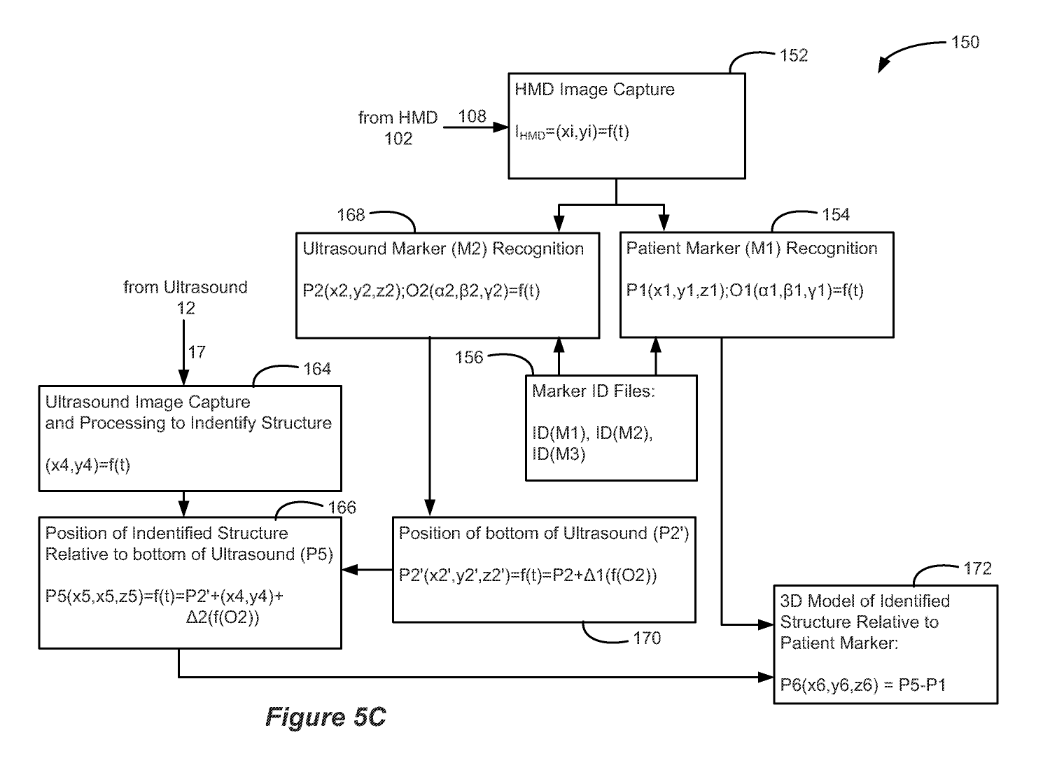

FIGS. 5A, 5B and 5C illustrate imaging of the vessel 24, and the formation of a 3D model of the vessel 24. Although not shown, at this point the clinician will have informed the computer 150 through normal input means (mouse, keyboard, etc.) to start capturing images from the ultrasound 12 via cables 17. As shown in FIG. 5A, the transducer 18, tracked as discussed earlier, is placed against the patient's skin 22, and is moved along the vessel 24 in the direction of arrow 99. The computer 150 captures a series of images from the ultrasound at different points in time, which are processed (box 164, FIG. 5C) to identify the vessel 24. Such image processing can occur in several ways, and can involve traditional image processing techniques. For example, the captured pixels from the ultrasound 12, which comprise a grey-scale or intensity values as well as locations in the plane 20 (FIG. 1), can be filtered relative to a threshold. This ensures that only those pixels above the intensity threshold (and hopefully indicative of the vessel 24) remain. Such filtering is particularly useful in the processing of ultrasound images, as such images generally contain noise and other artifacts not indicative of the structure being imaged.

FIG. 5B illustrates the images captured by the computer 150 post-processing at different points in time (t1, t2, t3), with the vessel 24 now represented as a number of pixels (x4,y4) without grey scale. One way of identifying the structure of interest (the vessel 24) is also illustrated. As shown in the captured image at time t2, eight positions (demarked by x) around the perimeter of the vessel 24 have been identified by the computer 150, roughly at 45 degrees around the structure, which generally matches the circular nature of the vessel. This is merely exemplary; other structures of interest (e.g., tumors) not having predictable geometries could present more complex images. In fact, it may be necessary for the clinician to interface with the computer 150 to review the ultrasound images and identify the structure of interest at any given time, with the clinician (for example) using input means to the computer 150 to highlight, or tag, the structure of interest. It is not ultimately important to the disclosed technique the manner in which the computer 150 filters and identifies the structure of interest in each of the ultrasound images, and other techniques could be used. Software useful for receiving and processing the images from the ultrasound in box 164 includes OpenCV, as described in “OpenCV,” which was submitted with the above-incorporated '740 Application.

With perimeter positions identified in each of the filtered ultrasound images, a 3D model of the vessel 24 can be compiled in the computer 150. As shown to the right in FIG. 5B, this 3D model can comprise a shell or hull formed by connecting corresponding perimeter positions in each of the images to interpolate the position of the vessel 24 in locations where there is no data. Optical flow with temporal averaging can be useful in identifying the perimeter positions around the post processed images and integrating these images together to form the 3D model. Optical flow is described in “Optical flow,” which was submitted with the above-incorporated '740 Application.

It is important that the 3D model of the vessel 24 be referenced to the patient marker, i.e., that the position of the 3D model to the patient marker M1 be fixed so that its virtual image can be properly viewed relative to the patient. Correctly fixing the position of the 3D model requires consideration of geometries present in the system 100. For example, while the tracked position and orientation of the transducer marker M2 (P2, O2) generally inform about the position of the vessel 24, the critical position to which the ultrasound images are referenced is the bottom center of the transducer 18, i.e., position P2′. As shown in FIG. 5A, the relation between P2 (the transducer marker M2) and the transducer bottom point P2′ is dictated by a vector, 41, whose length and angle are a function of the size of the transducer 18 and the particular position where the marker M2 is placed, and the orientation 02 of the transducer 18. Because the length and angle of 41 can be known before hand, and programmed into the computer 150, and because O2 is measured as a function of time, the orientation-less position of P2′ (x2′,y2′z2′) as a function of time can be calculated (box 170, FIG. 5C).

Another geometrical consideration is the relative position of the identified structure in each ultrasound image. For example, in the different time slices in FIG. 5B, it is seen that the position of the identified structure moves around in the image relative to the top center of the image where the bottom point of the transducer (P2′) is located. Such movement may be due to the fact that the identified structure is moving (turning) as the transducer 18 is moved over it, or could occur because the transducer (i.e., P2′) has not been moved in a perfectly straight line, as shown to the right in FIG. 5B.

To differentiate such possibilities, another vector, Δ2, is considered in each image that fixes the true position of the identified structure relative to the bottom point of the transducer (P2′). Calculation of Δ2 can occur in different manners in the computer 150. In the example shown in FIG. 5B, the computer 150 assesses the pixels (x4,y4) in each frame and computes a centroid C for each, which fixes the length and relative angle of Δ2 in each image. Δ2 in real space is also a function of the orientation O2 of the transducer 18—it cannot safely be assumed for example that the transducer 18 was held perfectly perpendicular to the skin 22 at each instance an ultrasound image is taken. By consideration of such factors, the 3D position of the identified structure relative to the bottom point of the transducer, P5(x5,y5,z5), comprises the sum of the position of that bottom point P2′, the vector Δ2, and the filtered pixels in each image (x4,y4) (box 166, FIG. 5C).

As noted earlier, it is important that the 3D model of the identified structure be related to the position of the patient marker M1. During image capture, both the position of the bottom transducer point (P2′) and the position of the patient marker M1 (P1) will move relative to the origin of the camera 104 in the HMD 102, as shown to the right in FIG. 5B. (In reality, the patient may be relatively still, but the HMD 102, i.e., the clinician's head, moves). To properly fix the 3D model of the structure relative to the patient marker M1, the position of M1, P1(x1,x2,x3) is subtracted from the 3D position of the identified structure relative to the bottom point of the transducer, P5(x5,y5,z5) (box 172, FIG. 5C). Both of these parameters P1 and P5 vary in time, and their subtraction yields a time-invariant set of points in 3D space relative to the patient marker M1, i.e., P6 (x6,y6,z6). The relevant points in P6 may also be supplemented by interpolation to form a 3D shell that connects corresponding perimeter positions, as discussed earlier with respect to FIG. 5B.

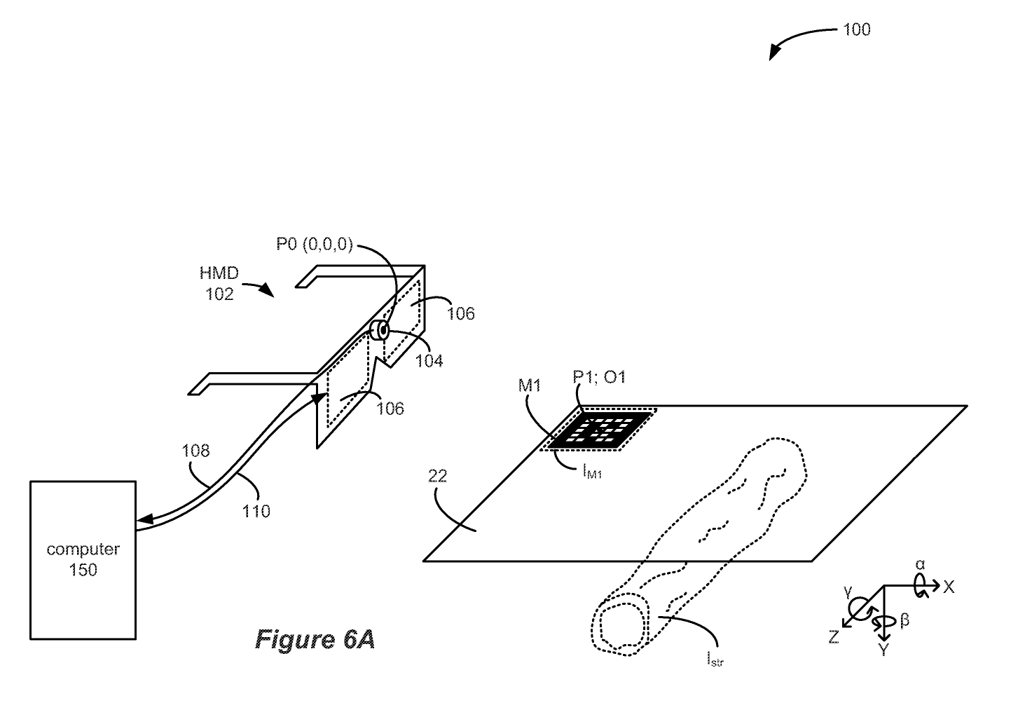

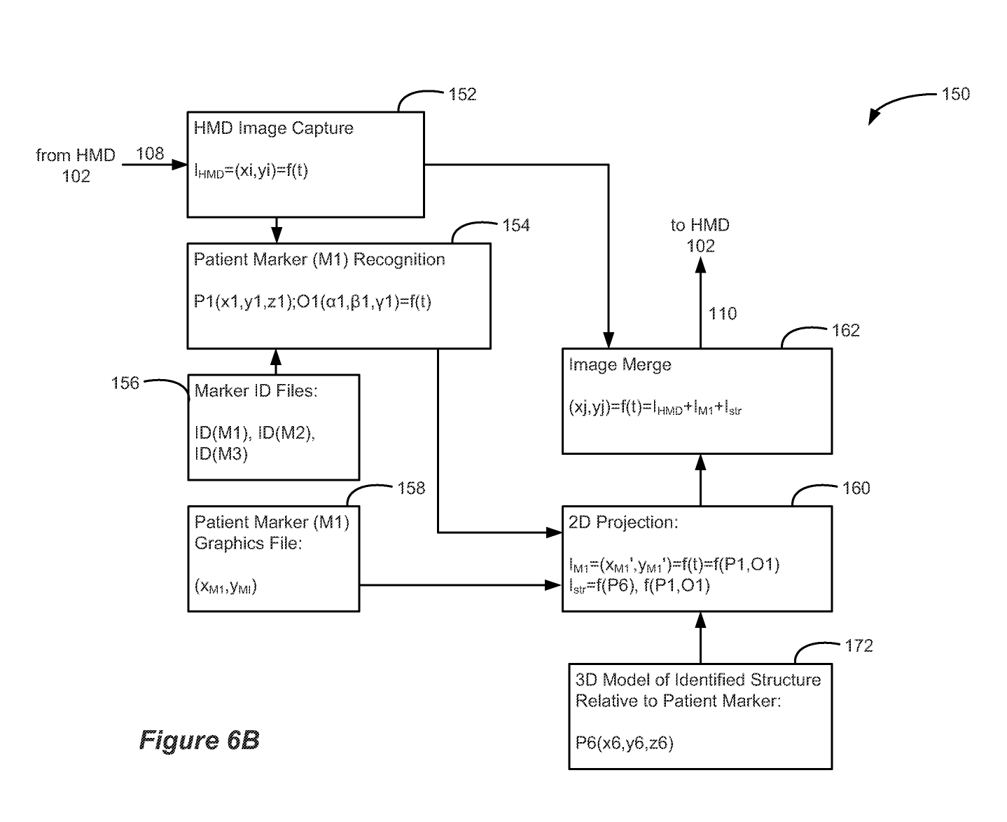

After compilation of the 3D model of the structure relative to the patient marker M1 is complete, the ultrasound 12 can be removed from the system 100, and the 3D model can be viewed through the HMD 102, as shown in FIGS. 6A and 6B. The position and orientation of the patient marker M1 is still optically tracked, and its virtual image, I_{M1}, is still visible and merged with live images, I_{HMD}, as similar boxes in FIG. 6B reflect. An image of the 3D model of the identified structure, Iₛₜᵣ, is also merged. To create the 2D projection of the 3D model, both the position of the model relative to the patient marker (P6), and the current position P1 and orientation O1 of the patient marker are considered. Thus, as the HMD 102 moves, Iₛₜᵣ will also change in size and orientation. In essence, the clinician can now virtually “see” the structure in proper perspective to the patient, although in reality that structure is beneath the skin 22 and not visible. Other information about the 3D model of the identified structure may also be indicated to the clinician, such as the size (e.g., width, length, or volume) of the model as calculated by the computer 150. Such other information may be output using the computer 150's traditional peripheral devices, or may be merged into the output image and displayed on the HMD 102.

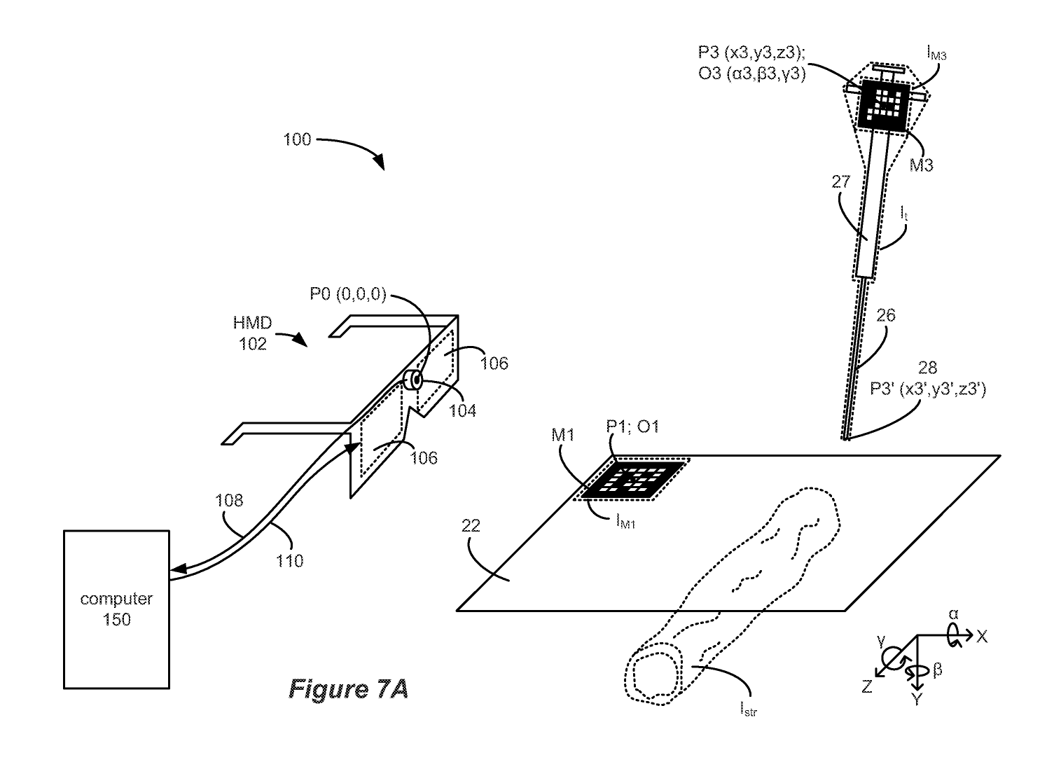

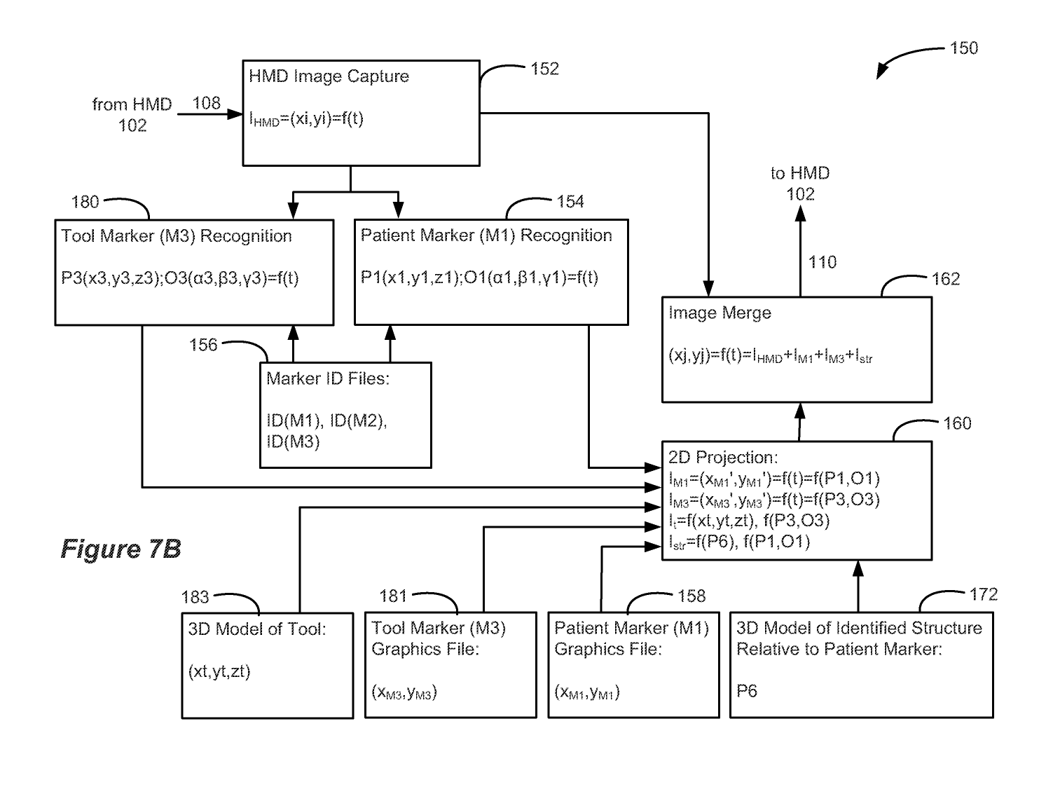

With this virtual image Iₛₜᵣ of the structure now in view, the clinician can introduce the tool 27 (e.g., needle 26) that will interact with that structure, which is shown in FIGS. 7A and 7B. A similar optically-detectable marker M3 is attached to the tool 27 with its own unique ID code (ID(M3)) encoded in its pattern of squares. As with the patient marker M1 and the transducer marker M2, the position P3(x3,y3,z3) and orientation O3(α3,β3,γ3) of the tool marker M3 relative to the camera 104 are recognized by the computer 150 (box 180, FIG. 7B). And again, a 2D virtual image of the tool marker M3, I_{M3}, is created and output to the displays 106 by reading a graphical file of the marker (x_{M3}, y_{M3}), and creating a 2D projection (x_{M3}′,y_{M3}′) (boxes 181, 160). This virtual image I_{M3} of tool marker M3 is a function of both the position P3 and orientation O3 of the tool marker M3 relative to the camera 104, and like image I_{M1} will change size and orientation as the HMD 102 moves. Merging of the tool marker image I_{M3} with both the patient marker image I_{M1} and the live images I_{HMD} (box 162) lets the clinician know that the tool is tracked.

Additionally beneficial at this stage, but not strictly necessary, is to provide a virtual image of the tool 27 itself, Iₜ, as shown in FIG. 7A. This is helpful for a number of reasons. First, viewing the tool virtually allows its perspective relative to the structure image, Iₛₜᵣ, to be better understood. For example, if the tool 27 is between the image of the structure and the HMD 102, Iₛₜᵣ should not be visible behind Iₜ, which gives the clinician a more natural perspective of the two images. Also, providing a virtual image Iₜ of the tool 27 is helpful in understanding the position of the tool 27 once it is no longer visible, e.g., when the needle 26 has been inserted into the patient. Because Iₜ shows the full length of the needle 26 even after it is placed in the patient, the relationship between its tip 28 and the virtual structure Iₛₜᵣ can be seen, even though neither are actually visible. This helps the clinician know when the needle tip 28 has breached the vessel 24, which as noted earlier is desirable when inserting an IV for example.

Creation of tool virtual image Iₜ starts with a file in the computer 150 indicative of the shape of the tool 27, which like the 3D model of the structure can comprise many points in 3D space, (xt,yt,zt) (box 183, FIG. 7B). This tool file (xt,yt,zt) can be made by optically scanning the tool, as an output of the Computer Aided Design (CAD) program used to design the tool, or simply by measuring the various dimensions of the tool. How the 3D tool file is created is not important, nor is it important that the tool image Iₜ produced from this file look exactly like the tool 27 in question. For example, (xt,yt,zt) and Iₜ may simply define and virtually display tool 27 as a straight rod of an appropriate length and diameter.

Tool image Iₜ, like the 3D model of the structure, can be rendered in 2D for eventual image merging and output to the displays 106 in the HMD 102 (box 160, FIG. 7B). Such 2D projection will be a function of the points (xt,yt,zt) projected in accordance with the position P3 and orientation O3 of the tool 27. For proper rendering, the position of the tool marker P3 on the tool 27 must also be known to the computer 150, as this position P3 will ultimately act as the origin of the projection of the tool. As with the other virtual images, the virtual image of the tool Iₜ will move and turn as either the HMD 102 or tool 27 moves and turns.

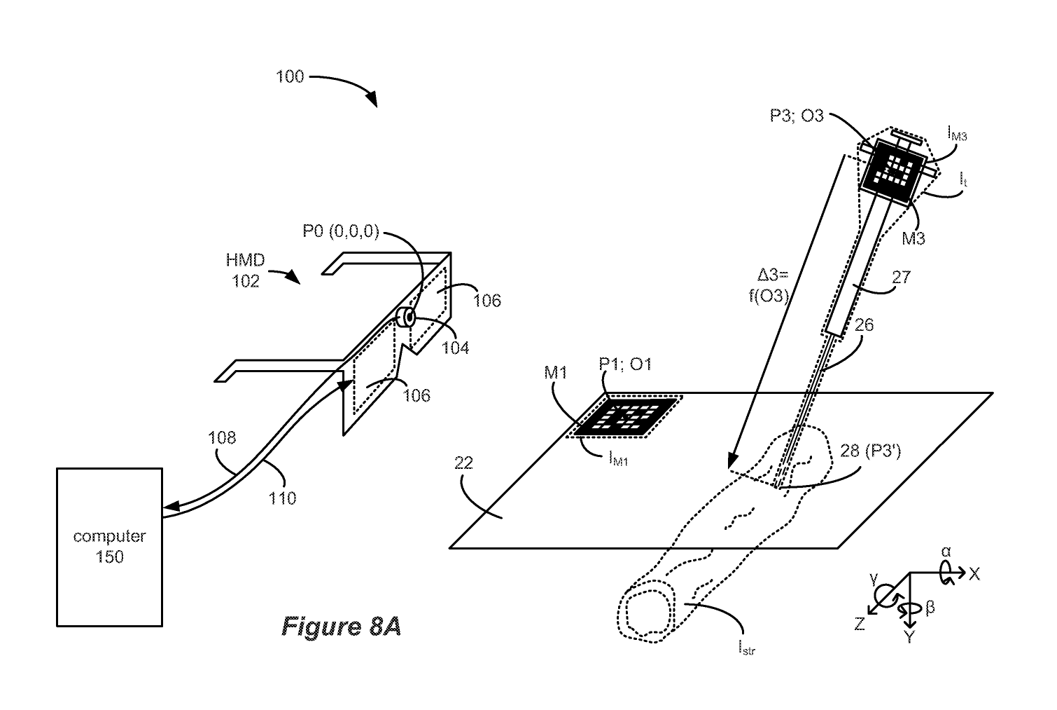

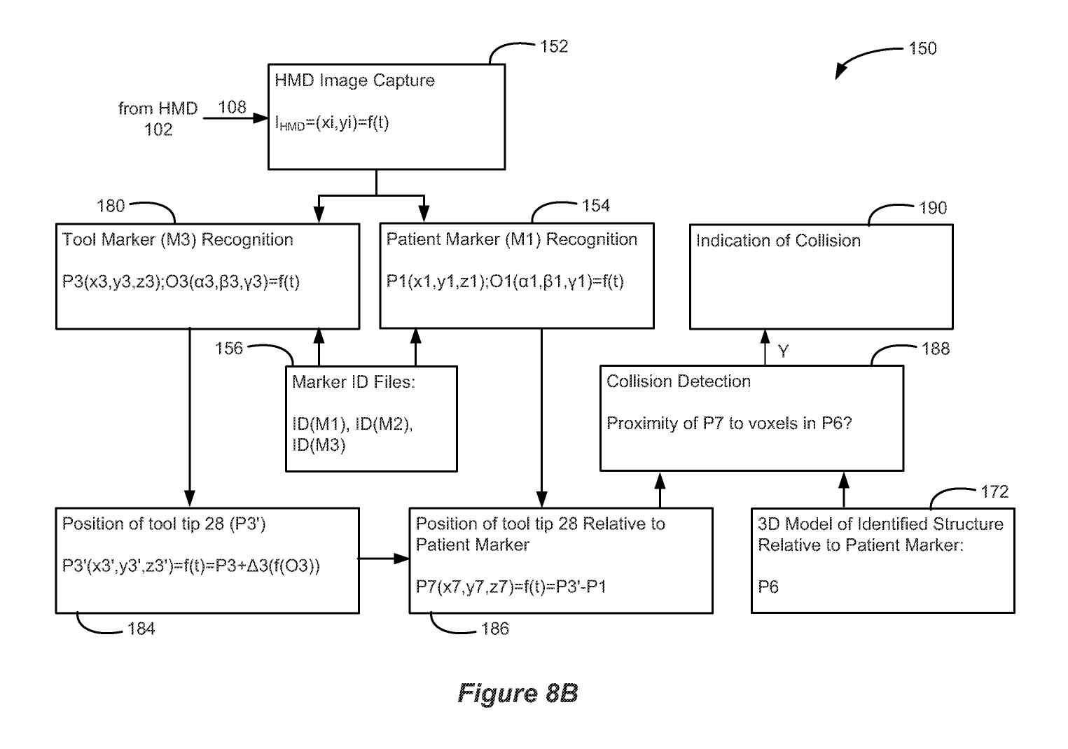

Once the virtual image of the tool 27 (Iₜ) and the virtual image of the structure (Iₛₜᵣ) are in viewed and properly tracked, the clinician may now introduce the tool 27 (needle 26) into the skin 22 of the patient, as shown in FIGS. 8A and 8B. As noted earlier, because the tool image Iₜ and structure image Iₛₜᵣ can be virtually seen beneath the skin 22 of the patient, the clinician can visually verify when the needle 26 has breached the vessel 24.

Additionally, the computer 150 can also automatically determine the proximity between the needle 26 and the vessel 24, which again requires consideration of the geometry present. The position of the needle tip 28, P3’, and the position of the tool marker, P3, are related by a vector 43, as shown in FIG. 8A. As with the position of the transducer marker (P2) relative to the bottom of the transducer (P2′), 43's length and angle are a function of the size of the tool 27, the particular position in which the tool marker M3 is placed, and the orientation O3 of the tool 27. Because the length and angle of Δ3 can be known before hand, and programmed into the computer 150, and because O3 is measured as a function of time, the orientation-less position of P3′ (x3′,y3′z3′) as a function of time can be calculated (box 184, FIG. 8B).

Because the position of the 3D model of the identified structure is referenced to the patient marker (P6; see box 172, FIG. 5C), it is also useful to reference the position of the needle tip 28 P3′ to the patient marker, which occurs by subtracting the current patient marker position P1 from the current position of the needle tip P3′, thus forming a normalized position for the tip, P7 (box 186, FIG. 8B). With positions P7 and P6 both referenced to the patient marker, the computer 150 can assess the proximity of the two by comparing P7 (in this case of a needle tip, a single point) to the pixels in P6 (collision detection box 188, FIG. 8B). This can occur by assessing in real time the minimum distance between P7 and the pixels in P6, or the shell formed by interpolating between the points in P6 as mentioned earlier. Such distance calculation is easily accomplished in many known ways.

In the event of a collision between P7 and P6, i.e., when the distance between them is zero, the computer 150 can indicate the collision (box 190, FIG. 8B) so that the clinician can know when the tip 28 has penetrated the vessel 24. Such indication can be accomplished using peripherals typically used with computer 150, such as sounds through speakers, indication on a computer system display, etc. Additionally, some other graphical indication of collision can be superimposed on the displays 106 of the HMD 102.

One skilled will understand that the system 100 is not limited to detecting collisions between the tool and the structure of interest. Using the same distance measurement techniques, the system can indicate relative degrees of proximity between the two. In some applications, it may be desired that the tool not breach the structure of interest, but instead merely get as close as possible thereto. Simple changes to the software of the collision detection module 188 (FIG. 8B) will allow for such modifications.

Further it is not necessary that collision of the tool be determined by reference to a single point on the tool, such as P7. In more complicated tool geometries, collision (or proximity more generally) can be assessed by comparing the position of the shell of the tool (such as represented by the 3D model of the tool; see box 183, FIG. 7B) versus the shell of the imaged structure.

It should be understood that while this disclosure has focused on the example of positioning a needle tip within a vessel, it is not so limited. Instead, the disclosed system can be varied and used in many different types of medical procedures, each involving different structures of interest, different tools, and different forms of imaging. Furthermore, the use of ultrasound, while preferred as an imaging tool for its quick and easy ability to image structures in situ and in real time during a procedure, is not necessary. Other forms of imaging, including those preceding the medical procedure at hand, can also be used, with the resulting images being positionally referenced to the patient in various ways.

The imaging device may not necessarily produce a plurality of images for the computer to assess. Instead, a single image can be used, which by its nature provides a 3D model of the structure of interest to the computer 150. Even a single 2D image of the structure of interest can be used. While such an application would not inform the computer 150 of the full 3D nature of the structure of interest, such a single 2D image would still allow the computer to determine proximity of the tool 27 to the structure of interest.

While optical tracking has been disclosed as a preferred manner for determining the relative positions and orientations of the various aspects of the system (the patient, the imaging device, the tool, etc.), other means for making these determinations are also possible. For example, the HMD, patient, imaging device, and tool can be tagged with radio transceivers for wirelessly calculating the distance between the HMD and the other components, and 3-axis accelerometers to determine and wirelessly transmit orientation information to the HMD. If such electrical markers are used, optical marker recognition would not be necessary, but the clinician could still use the HMD to view the relevant virtual images. Instead, the electronic markers could be sensed wirelessly, either at the computer 150 (which would assume the computer 150 acts as the origin of the system 100, in which case the position and orientation of the HMD 102 would also need to be tracked) or at the HMD 102 (if the HMD 102 continues to act as the origin).

Software aspects of the system can be integrated into a single program for use by the clinician in the procedure room. As is typical, the clinician can run the program by interfacing with the computer 150 using well known means (keyboard, mouse, graphical user interface). The program can instruct the clinician through the illustrated process. For example, the software can prompt the clinician to enter certain relevant parameters, such the type of imaging device and tool being used, their sizes (as might be relevant to determined vectors Δ1, Δ2, Δ3 for example), and the locations of the relevant marker images and 3D tool files (if not already known). The program can further prompt the clinician to put on the HMD 102, to mark the patient, and confirm that patient marker is being tracked. The program can then prompt the clinician to mark the transducer (if not already marked), and confirm that the transducer marker is being tracked. The clinician can then select an option in the program to allow the computer 150 to start receiving and processing images from the ultrasound 12, at which point the clinician can move the transducer to image the structure, and then inform the program when image capture can stop. The program could allow the clinician to manually review the post-processed (filtered) images to confirm that the correct structure has been identified, and that the resulting 3D model of the imaged structure seems to be appropriate. The program can then display the 3D model of the structure through the HMD 102, and prompt the clinician to mark the tool (if not already marked), and confirm that the tool marker is being tracked. The program can then inform the clinician to insert the tool into the patient, and to ultimately indicate the proximity of the tool to the structure, as already discussed above. Not all of these steps would be necessary in a computer program for practicing the process enabled by system 100, and many modifications are possible.

One skilled in the art will understand that the data manipulation provided in the various boxes in the Figures can be performed in computer 150 in various ways, and that various pre-existing software modules or libraries such as those mentioned earlier can be useful. Other data processing aspects can be written in any suitable computer code, such as Python.

The software aspects of system 100 can be embodied in computer-readable media, such as a single medium or multiple media (e.g., a centralized or distributed database, and/or associated caches and servers) that store instructions for execution by a machine, such as the computer system 15 disclosed earlier. Examples of computer-readable media include, but are not limited to, solid-state memories, or optical or magnetic media such as discs. Software for the system 100 can also be implemented in digital electronic circuitry, in computer hardware, in firmware, in special purpose logic circuitry such as an FPGA (field programmable gate array) or an ASIC (application-specific integrated circuit), in software, or in combinations of them, which again all comprise examples of “computer-readable media.” When implemented as software fixed in computer-readable media, such software can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Computer 150 should be understood accordingly, although computer 150 can also comprise typical work stations or personal computers.

Routine calibration of the system 100 can be useful. For example, it can be useful to place one of the markers at a known distance from the camera 104, and to assess the position that the computer 150 determines. If the position differs from the known distance, the software can be calibrated accordingly. Orientation can be similarly calibrated by placing a marker at a known orientation, and assessing orientation in the computer to see if adjustments are necessary.

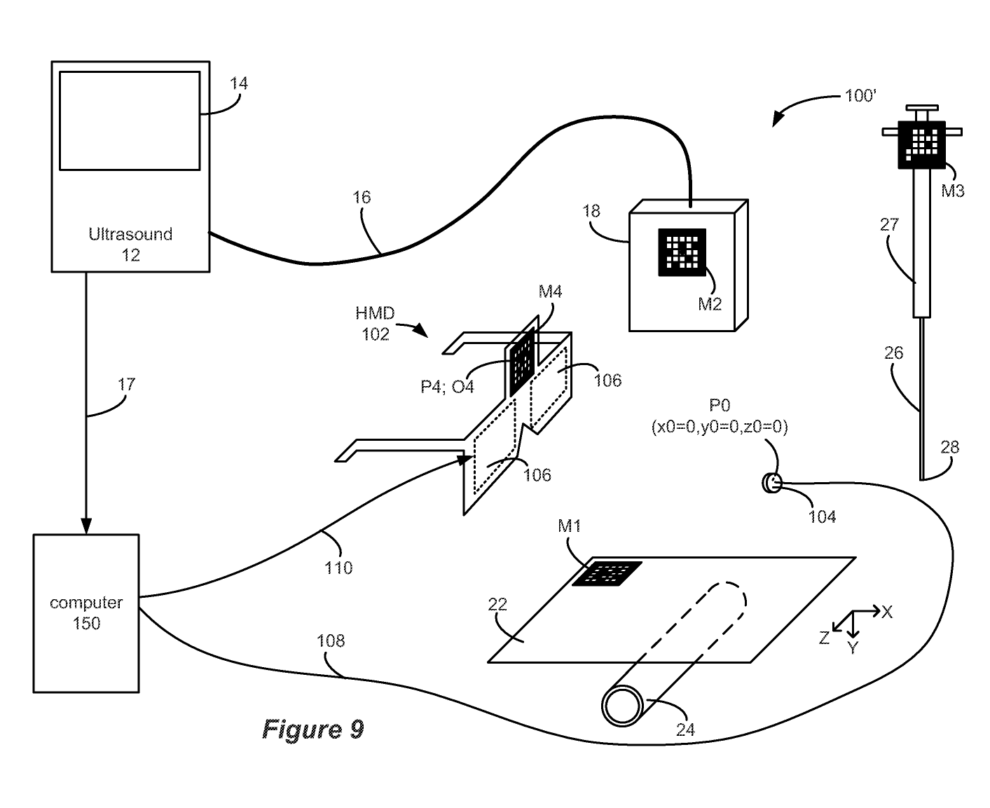

FIG. 9 illustrates another example of an improved system 100′ in which the camera 104 is separated from the HMD 102. In this system, the camera 104 would likely be positioned in some stationary manner relative to the patient, and able to view the other components of the system 100′. (It is not however strictly required that the camera be stationary, as system 100′ can adjust to camera 104 movement). The camera 104 can still act as the origin (P0) of the system, against which the position and orientation of the various other components—the patient (P1;O1), the ultrasound transducer 18 (P2;O2), the tool 27 (P3;O3), and now the HMD 102 (P4;O4) which is marked with marker M4—are gauged. Because position and orientation of the HMD 102 is now tracked relative to the camera 104, the HMD 102 also comprises a marker M4, for which a corresponding HMD marker image I_{M4} is stored in the computer 150.

As before, the HMD 102 in system 100′ can be of the opaque or the optical see through type. If the HMD 102 is of the opaque type, the HMD 102 would have another image capture device (i.e., another camera apart from stationary camera 104) to capture the clinician's view (I_{HMD}) so that it can be overlaid with other images (the markers, the ultrasound, the tool, etc.) as described above. However, as illustrated in FIG. 9, the displays 106 in the HMD 102 are at least semi-transparent, and as such live images don't need to be captured by the HMD 102 and merged with other system images before presentation at the displays 106.

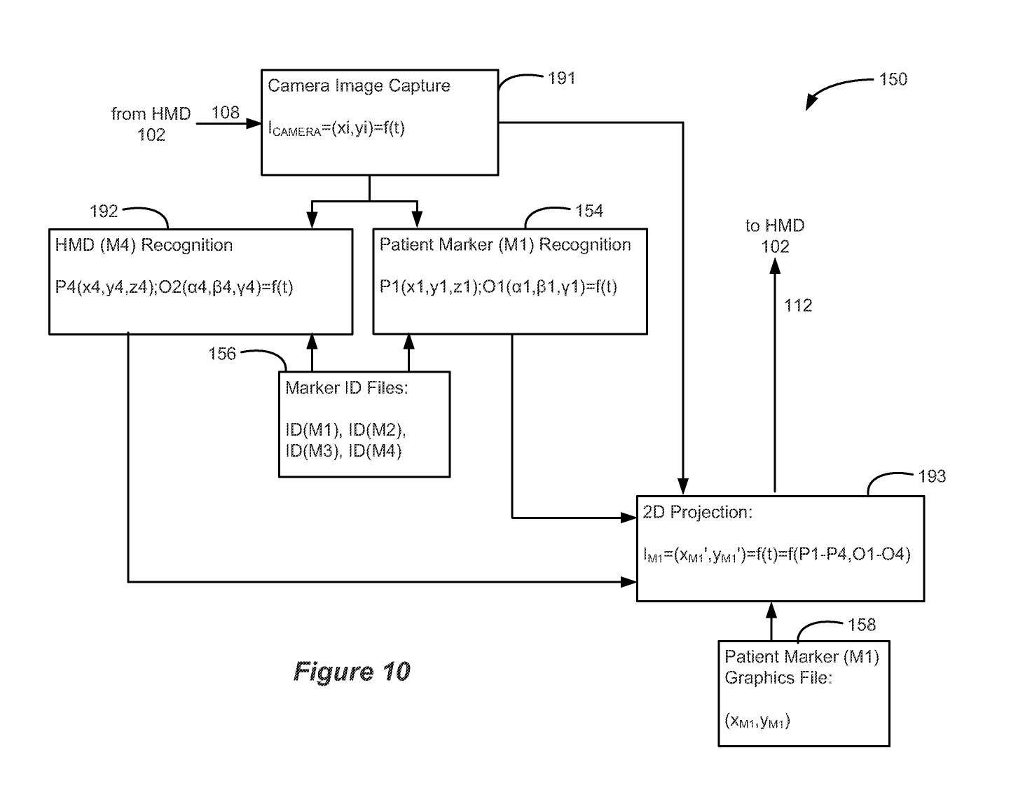

System 100′ can otherwise generally operate as described earlier, with some modifications in light of the new origin of the camera 104 apart from the HMD 102, and in light of the fact that the clinician's view is not being captured for overlay purposes. For example, FIG. 10 shows use of the system 100′ in an initial step—i.e., prior to the introduction of the ultrasound transducer 18 as in FIGS. 3A and 3B. At this step in system 100′, the camera 104 captures an image (191), and the position and orientation of the patient marker M1 (P1;O1) and the HMD marker M4 (P4;O4) are identified (steps 154 and 191). From these, step 193 can create a 2D projection (IM1) of the patient marker M1 from graphics file 158 for presentation to the display of the HMD 102. (There is no need for an image of the HMD marker M4, because the clinician would not see this). Because this image is to be displayed at the position of the HMD marker M4, the position and orientation of HMD marker M4 are subtracted from position and orientation of the patient marker M1 at step 193. As this 2D image IM1 will be displayed on the displays 106 without overlay of the clinician's view, there is no need in this example for image merging (compare step 162, FIG. 3B), although if a separate image capture device is associated with the HMD 102, such merging would occur as before. Other steps in the process would be similarly revised in light of the new position of the camera 104, as one skilled in the art will appreciate.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system useful in performing a medical procedure on a patient, comprising:
a computer;
a display;
a patient marker affixable to a patient, wherein the patient marker informs the computer of a position and orientation of the patient marker;
an imaging device marker affixable to an imaging device, wherein the imaging device marker informs the computer of a position and orientation of the imaging device marker;
a tool marker affixable to a tool for interfacing with a structure of interest in the patient, wherein the tool marker informs the computer of a position and orientation of the tool marker;
wherein the computer is configured to receive at least one image of the structure of interest from the imaging device,
wherein the computer is configured to generate a 3D model of the structure of interest using the at least one image,
wherein the computer is configured to generate a virtual image of the structure of interest from the 3D model of the structure of interest, and to generate a virtual image of the tool from a 3D model indicative of the shape of the tool, and
wherein the computer is configured to superimpose the virtual image of the structure of interest and the virtual image of the tool on the display in correct positions and orientations relative to the patient.

2. The system of claim 1, wherein the display comprises a head mounted display (HMD).

3. The system of claim 2, wherein the HMD further comprises a camera for capturing live images.

4. The system of claim 3, wherein the patient marker, the imaging device marker, and the tool marker are optical markers, and wherein the optical markers are sensed by the camera to inform the computer of their positions and orientations.

5. The system of claim 3, wherein the live images are sent to the computer by the camera, wherein the computer is configured to superimpose the virtual image of the structure of interest, the virtual image of the tool, and the live images on the display in correct positions and orientations relative to the patient.

6. The system of claim 2, wherein the HMD is at least semi-transparent such that the HMD allows the user to view the live images through the HMD.

7. The system of claim 1, wherein the patient marker, the imaging device marker, and the tool marker are electronic markers, and wherein the position and orientation of the electronic markers are sensed wirelessly.

8. The system of claim 1, further comprising a camera, wherein the patient marker, the imaging device marker, and the tool marker are optical markers, and wherein the optical markers are sensed by the camera to inform the computer of their positions and orientations.

9. The system of claim 8, wherein the camera is coupled to the display.

10. The system of claim 8, wherein the camera is separate from the display.

11. The system of claim 8, wherein the camera sends live images to the computer, wherein the computer is further configured to superimpose a virtual image of at least one of the patient, imaging device, or tool markers on the live images in the HMD in correct positions and orientations relative to the patient.

12. The system of claim 1, wherein the computer is further configured to determine a proximity between the virtual image of the structure of interest and the virtual image of the tool.

13. The system of claim 1, wherein the computer is further configured to determine a collision between the virtual image of the structure of interest and the virtual image of the tool.

14. The system of claim 13, wherein the computer is further configured to indicate the collision to the user.

15. The system of claim 149, wherein the computer is further configured to alert the user of the collision by displaying an image on the display.

16. The system of claim 1, wherein the at least one image comprises a plurality of images.

17. The system of claim 16, wherein the computer is configured to generate the 3D model of the structure by determining perimeter positions of the structure of interest in each image, and connecting corresponding perimeter positions in each images.

18. A system useful in performing a medical procedure on a patient using a tool, comprising:
a computer;
a patient marker affixable to a patient, wherein the patient marker informs the computer of a position and orientation of the patient marker;
an imaging device marker affixable to an imaging device, wherein the imaging device marker informs the computer of a position and orientation of the imaging device marker;
a tool marker affixable to a tool for interfacing with a structure of interest in the patient, wherein the tool marker informs the computer of a position and orientation of the tool relative to the patient;
wherein the computer is configured to receive at least one image of the structure of interest from the imaging device,
wherein the computer is configured to generate a 3D model of the structure of interest positioned relative to the patient using the at least one image, and
wherein the computer is configured to determine a proximity between the 3D model of the structure of interest and the tool.

19. The system of claim 18, wherein the computer is configured to determine a proximity between the 3D model of the structure of interest and the tool by calculating a distance between the 3D model of the structure of interest positioned relative to the patient and a point on the tool positioned relative to the patient.

20. The system of claim 18, wherein the computer is further configured to generate a virtual image of the structure of interest from the 3D model of the structure of interest, and to generate a virtual image of the tool from a 3D model indicative of the shape of the tool.

21. The system of claim 20, wherein the computer is configured to determine a proximity between the 3D model of the structure of interest and the tool by calculating a distance between the virtual image of the structure of interest and the virtual image of the tool.

22. The system of claim 20, further comprising a display device, wherein the computer is further configured to superimpose the virtual image of the structure of interest and the virtual image of the tool on the display device in correct positions and orientations relative to the patient.

23. The system of claim 22, wherein the display device comprises a head mounted display (HMD).

24. The system of claim 23, wherein the HMD is opaque, and wherein live images are sent to the computer by a camera on the HMD and are provided from the computer to the HMD.

25. The system of claim 23, wherein the HMD is at least semi-transparent such that the HMD allowing a user to view live images through the HMD.

26. The system of claim 22, further comprising a camera, and wherein the patient marker, the imaging device marker, and the tool marker are optical markers, and wherein the optical markers are sensed by the camera to inform the computer of their positions and orientations.

27. The system of claim 26, wherein the camera is coupled to a display.

28. The system of claim 27, wherein the camera sends live images to the computer, wherein the computer is further configured to superimpose a virtual image of at least one of the patient, imaging device, or tool markers on the live images in the display in correct positions and orientations relative to the patient.

29. The system of claim 18, wherein the patient marker, the imaging device marker, and the tool marker are electronic markers, and wherein the position and orientation of the electronic markers are sensed wirelessly.

30. The system of claim 18, wherein the proximity comprises a collision between the 3D model of the structure of interest and the tool.

31. The system of claim 30, wherein the computer is further configured to indicate the collision to a user.

32. The system of claim 18, wherein the at least one image comprises a plurality of images.

33. The system of claim 32, wherein the computer is configured to generate the 3D model of the structure by determining perimeter positions of the structure of interest in each image, and connecting corresponding perimeter positions in each images.
